(19) United States
(12) Patent Application Publication
(10) Pub. No.: US 2016/0029926 A1
(43) Pub. Date: Feb. 4, 2016

(54) ORTHOPEDIC LOAD LIMIT WARNING APPARATUS

(71) Applicant: Gerald G. Varnum, Pinckneyville, IL (US)

(72) Inventor: Gerald G. Varnum, Pinckneyville, IL (US)

(21) Appl. No.: 14/542,094

(22) Filed: Nov. 14, 2014

Related U.S. Application Data
(60) Provisional application No. 61/999,509, filed on Jul. 29, 2014.

(51) Int. Cl. A61B 5/103; G08B 21/18; A43B 3/00

(52) U.S. Cl. CPC A61B 5/1036; A43B 3/0005; G08B 21/182

(57) ABSTRACT

An orthopedic load limit warning apparatus having a pressure sensor shaped and sized for attachment to the underside of a user's lower appendage, the pressure sensor detecting and making a measurement of the load placed upon the sensor by the user, a pressure switch operatively associated with the pressure sensor, the pressure sensor communicating the load measurement to the pressure switch, and a sensory alarm connected to the pressure switch, the pressure switch selectively activating the sensory alarm to produce a sensory warning, the pressure sensor activating the sensory alarm when the load exceeds a predetermined level.

### CROSS-REFERENCE TO RELATED APPLICATIONS

This application derives and claims priority from U.S. provisional application 61/999,509 filed Jul. 29, 2014, which application is incorporated herein by reference.

### STATEMENT REGARDING FEDERALLY SPONSORED RESEARCH OR DEVELOPMENT

Not applicable.

### FIELD OF THE INVENTION

The present disclosure relates to an orthotic load limit detection and warning apparatus, and more specifically, to a device adapted to sense a load placed upon an individual's lower appendage and convey that information to the individual with a warning when the load exceeds a predetermined level.

### BACKGROUND OF THE INVENTION

The statements in this section merely provide background information related to the present disclosure and may not constitute prior art.

When healthy, an individual's lower appendages are capable of supporting substantial loads. These include loads regularly imparted on the lower appendages from non-strenuous daily activities, such as for example, standing, lowering or raising the body to and from a seated position, and walking. In addition, healthy lower appendages are structured to withstand loads brought about by more strenuous activities such as jumping, running, and carrying loads in addition to the load of the individual's body weight. However, when one or more of an individual's lower appendages is injured or for some other reason has become capable of supporting no more than a predetermined load limit that is less than the load the appendage should be able to support when healthy, exceeding the load limit can result in damage or further injury to the individual's lower appendage and quite possibly to other parts of the individual's body due to a fall or a resulting compensational injury. It is therefore important for the individual to insure that the load limit is not exceeded.

Consequently, when treating a patient with an injured or weak lower appendage, a physician will often prescribe a weight or load limit for that lower appendage. Unfortunately, it is often difficult for a patient to correlate a prescribed load limit for a lower appendage with daily activities that may impart loads on that appendage. That is, a patient may understand that a load limit of thirty (30) pounds is not to be exceeded, but that same patient may not be able to readily discern when the load reaches thirty (30) pounds.

It would therefore be desirable to have an inconspicuous apparatus that measures the load imparted on a patient's lower appendage and conveys that load in real time to the patient. It would be further desirable for that device to provide a warning to the patient when the lower appendage load reaches or exceeds a predetermined level.

### BRIEF DESCRIPTION OF THE DRAWINGS

The illustrative embodiments of the present invention are shown in the following drawings which form a part of the specification:

FIG. 1 is a perspective view of the underside of a footwear weight limit warning apparatus incorporating the present disclosure according to one representative embodiment, the apparatus having a pressure sensor in the shape of a shoe sole insert, a pressure switch operatively attached to the heel of the sole insert, a sensory alarm attached to the pressure switch, and a battery connected to the pressure switch and the sensory alarm.

FIG. 2 is a perspective view of the topside of the footwear insert of FIG. 1.

FIG. 3 is a perspective view of the topside of the footwear insert of FIG. 1 shown in relation to a representative shoe, the shoe depicted in perspective view with the shoe upper shown in partial ghost.

FIG. 4 is a cutaway side view of the footwear insert of FIG. 1 placed in a shoe, the shoe having an opening in the heel to receive the electronics portion of the insert, the shoe heel and sole being shown in cutaway and the shoe upper being shown in ghost.

FIG. 5 is a side view of the footwear insert of FIG. 1 placed in a shoe, the shoe heel and sole being shown in cutaway and the shoe upper being shown in ghost.

FIG. 6 is a perspective view of an alternative embodiment of a footwear insert incorporating the present disclosure having the sensory alarm extended upward and attached to the user's leg above the ankle, the sole insert being positioned in a shoe, the shoe heel and sole being shown in partial cutaway and the shoe upper being shown in ghost.

FIG. 7 is a side view of yet another alternate embodiment of the sole insert incorporating the present disclosure having the pressure switch and battery communicate wirelessly with the sensory alarm or alternatively with an electronic device with a computer processor, such as for example, a cellular phone, the shoe heel and sole being shown in cutaway and the shoe upper being shown in ghost.

FIG. 8 is a representative schematic of certain components of the disclosure of FIGS. 1-6.

FIG. 9 is a representative schematic of certain components of the disclosure of FIG. 7.

FIG. 10 is a representative schematic of certain components of the disclosure of FIG. 7, further incorporating a relay for use with certain sensory alarms.

Corresponding reference characters indicate corresponding parts throughout the several views of the drawings.

### DETAILED DESCRIPTION

The following description is merely representative in nature and is not intended to limit the present disclosure or the disclosure's applications or uses. Before turning to the figures and the various representative embodiments illustrated therein, a detailed overview of various embodiments and aspects is provided for purposes of breadth of scope, context, clarity, and completeness. While the invention will be described and disclosed here in connection with certain preferred embodiments, the description is not intended to limit the invention to the specific embodiments shown and described here, but rather the invention is intended to cover all alternative embodiments and modifications that fall within the spirit and scope of the invention as defined by the claims included herein as well as any equivalents of the disclosed and claimed invention.

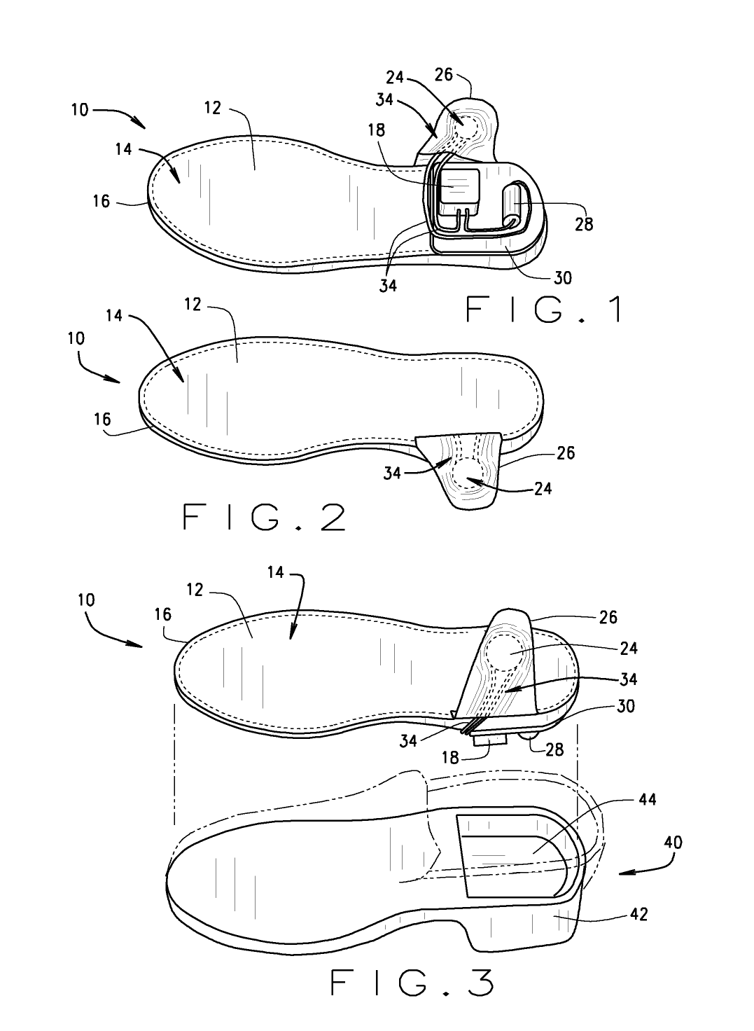

Referring now to FIGS. 1-9, an orthopedic load limit warning apparatus 10 having a variety of different components and embodiments is disclosed and illustrated. In one embodiment (FIGS. 1-5), the warning apparatus, generally referred to at 10, comprises a pressure actuation device or sensor in the form of a generally pliant and hollow footwear sole insert 12 that is filled with a compression fluid 14 and has an outer perimeter 16. The apparatus further comprises an adjustable electric pressure switch 18 operatively associated with the sole insert 12. The pressure switch 18 has a pressure port 20 (not shown) that is attached to an opening or port 22 (not shown) in the sole insert 12. Consequently, the fluid 14 flows between the sole insert 12 and the pressure switch 18, and the fluid pressure is substantially equal throughout fluid 14 in the sole insert 12 and the pressure switch 18. The apparatus 10 further includes a sensory alarm 24 housed in a soft, flexible wrap 26, and a battery 28 operatively connected to the pressure switch 18 and connected to and powering the sensory alarm 24.

The pressure switch 18 and battery 28 are securely attached to a support plate 30 which has the general shape of and is securely attached to the underside of the heel portion of the sole insert 12 between the sole insert 12 and the pressure switch 18. An opening or port 32 in the support plate 30 is aligned with the pressure port 20 to enable the pressure switch 18 to attach to the sole insert at the port 22 through the opening 32 in the support plate 30.

A set of wires 34 operatively attach to the pressure switch 18 and the battery 28 on the underside of the sole insert 12, extend outward to the perimeter 16 of the heel portion of the sole insert 12 where said wires 34 are encased in the flexible wrap 26, and extend yet further a short distance beyond the perimeter 16 to operatively attach to the sensory alarm 24. The wires 34 thereby operatively connect the sensory alarm 24 to the pressure switch 18 and the battery 28.

As can be appreciated, the portions of the wires 34 that extend beyond the perimeter 16 of the sole insert 12 provide substantial flexibility in the positioning of the sensory alarm 24 relative to the sole insert 12 so as to enable placement of the sensory alarm 24 against the lower leg of a user in a comfortable manner. That is, the sensory alarm 24 can be positioned in virtually any position next to the perimeter 16 from which the wires 34 extend, only limited by the length of the wires beyond the perimeter 16. Of course, it is recognized that the warning apparatus 10 may be designed such that the wires 34 may be of virtually any length to provide even greater flexibility for positioning the sensory alarm 24.

The sole insert 12 is shaped and sized to fit into a shoe upper atop the sole of the shoe, is generally flat and substantially hollow, and has a sealed pocket 38 that houses the compression fluid 14. The pocket 38 is likewise generally flat with an outer edge that tracks the contours of the perimeter 16 of the sole insert 12. Due to fluid dynamics, pressure applied to any portion of the pocket 38 will increase the pressure through all of the fluid 14 contained in the pocket 38. Hence, in this configuration of the warning apparatus 10, the pocket 38 and fluid 14 provide fluid pressure sensitivity across virtually the entire upper surface of the sole insert 12. That is, when the sole insert 12 is placed within a shoe or other form of footwear, and a user places a foot or other lower appendage within the footwear, the user's foot will transmit the user's weight load—such as when the user is standing, walking or running—as a force pushing down on the fluid 14 confined within the pocket 38 of the sole insert 12.

The battery 28 provides electric power to operate the sensory alarm 24 in conjunction with the pressure switch 18. The pressure switch 18 and the sole insert 12 are mutually configured to allow the pressure switch 18 to sense and monitor the pressure of the fluid 14 within the pocket 38 exerted by a user's lower appendage. This pressure is proportional to the weight supported on that foot or lower appendage. When the pressure switch 18 senses that the pressure of the fluid 14 within the pocket 38 exceeds a predetermined upper load limit corresponding to a maximum recommended weight limit, the pressure switch 18 closes, completing the circuit from the battery 28 to the sensory alarm 24. The sensory alarm 24 then activates to provide an audio, vibratory or other alert or warning to the user that the weight placed upon that lower appendage has exceeded the prescribed limit.

Thus, in operation, the user sets an upper load limit for the pressure switch 18 at a level desired by the user. The upper load limit will typically be a value prescribed by a physician, but the value can also be set for other purposes as well. The sole insert 12, the pressure switch 18 and the battery 28 are then placed within a shoe 40 or other footwear that is to be placed on the foot of the user that is subject to a weight limitation. Of course, the warning apparatus 10 can be configured to allow for setting and adjustment of the upper load limit after the sole insert 12, the pressure switch 18 and the battery 28 are placed within the shoe 40.

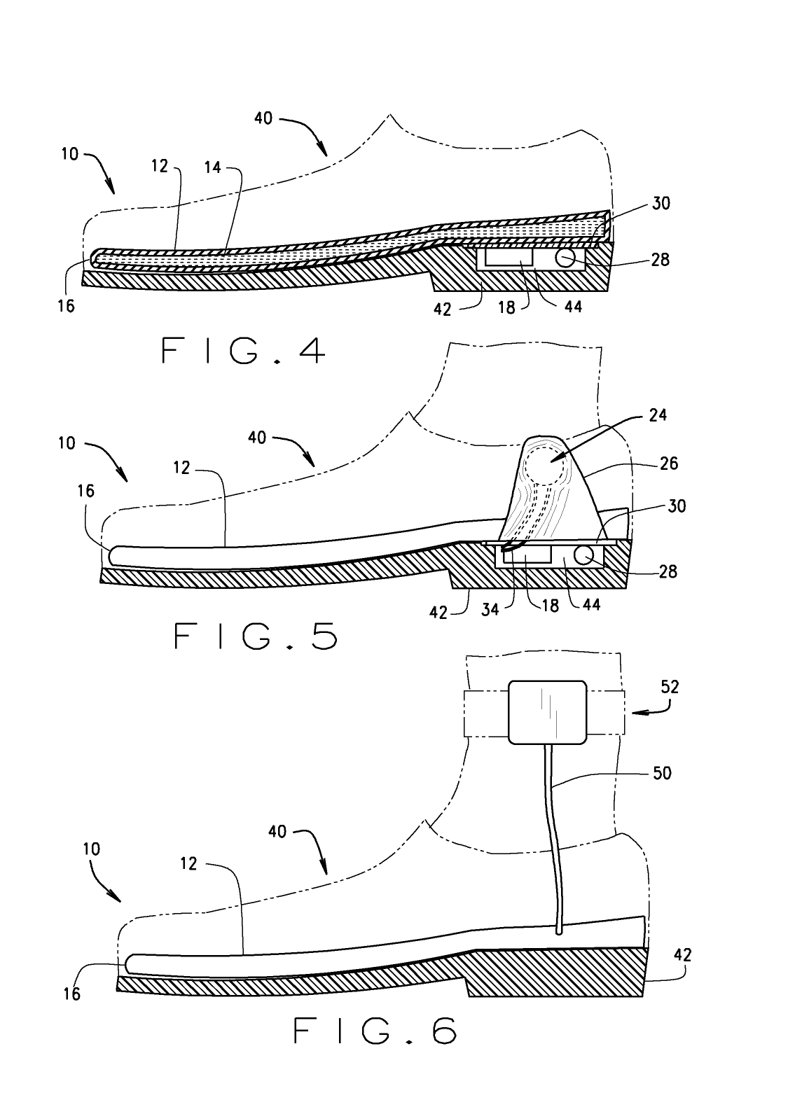

Referring to FIGS. 4 and 5, for the embodiment of the warning apparatus 10, the shoe 40 has a specially formed heel 42 with a cavity 44 opening upward through the upper face of the heel 42 that is shaped and sized to receive the lower electrical components of the warning apparatus 10, namely the pressure switch 18, the battery 28 and the portions of the wires 34 below the heel of the sole insert 12. When the warning apparatus 10 is properly placed in the shoe 40, with the support plate 30 positioned top the cavity 44, the heel portion of the sole insert 12 rests atop the support plate 30, and the pressure switch 14, the battery 20 and the portions of the wires 22 are all contained within the cavity 44 below the support plate 30. As can be seen in FIG. 5, placing the shoe 40 upon the foot (lower appendage) that is subject to a weight limitation allows the sensory alarm 24 to rest against the user's ankle. This, however, is a preferred arrangement, and the sensory alarm 24, the flexible wrap 26 and the wires 34 can be modified in other embodiments to allow for positioning of the sensory alarm 24 at virtually any location on the user's body. As can be appreciated, the relatively small size and flat profile of the warning apparatus 10 minimizes any potential discomfort caused by its use. Further, well-recognized size, shape and material variations for each of the components of the warning apparatus 10 can be utilized to even further reduce any potential discomfort.

With the warning apparatus 10 properly positioned in the shoe 40, and the shoe 40 placed upon a user's foot that is subject to a weight limitation, the user can then proceed with ambulatory function. Should the user place a load upon the foot that is subject to a weight limitation, the load will depress the sole insert 12, thereby increase the pressure of the fluid 14 within the pocket 38 of the sole insert 12, and trigger the pressure switch 18. The pressure switch 18 will then allow the battery 28 to activate the sensory alarm 24 to signal to the user that the upper load limit has been exceeded.

An alternate embodiment of the apparatus 10 is shown in FIG. 6. Here, the sole insert 12 can be placed in any shoe or other footwear that fits the user's lower appendage. A small hose 50 connects to the sole insert 12 and conducts a portion of the fluid 14 from the pocket 38, up the user's ankle to the pressure port 20 of the pressure switch 18. In this embodiment, the battery 28, pressure switch 18 and sensory alarm 24 are contained in an adjustable elastic strap assembly 52, which removably attaches to the user's lower leg just above the ankle. This provides substantial flexibility in the placement of the battery 28, pressure switch 18 and sensory alarm 24.

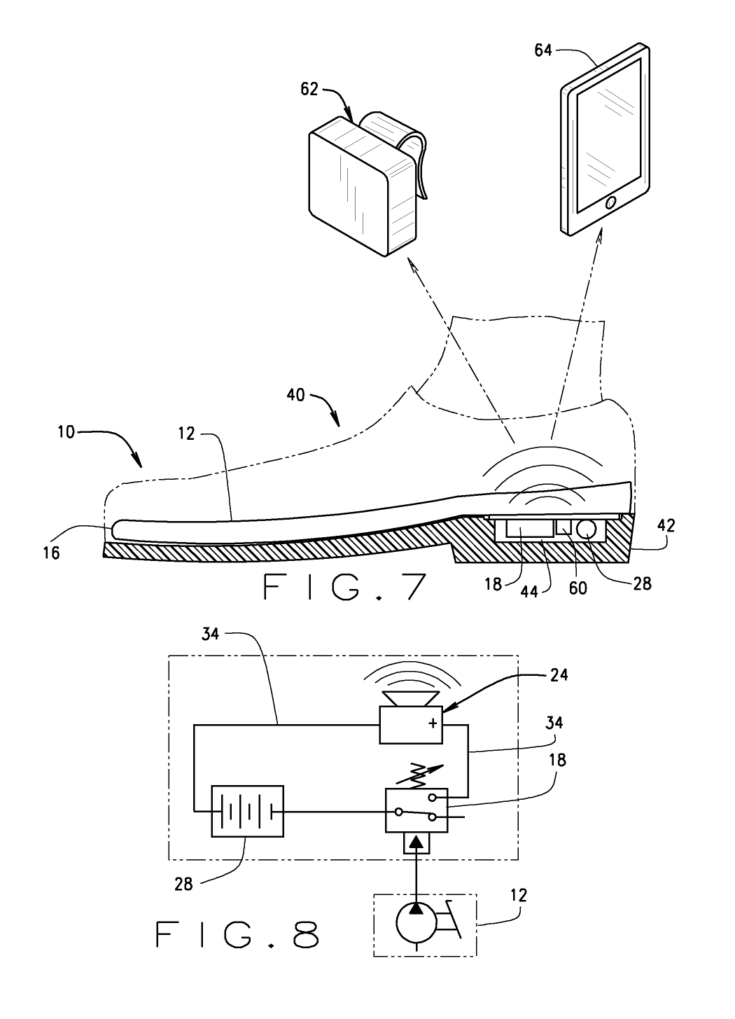

In yet another embodiment, FIG. 7 depicts the apparatus 10 having a pressure sensor sole insert 12, a pressure switch 18 and a battery 28, all positioned within cavity 44 of the heel 42 of shoe 40. The support plate 30 is positioned atop and sized to entirely cover the cavity 44. The pressure switch 18 and battery 28 are securely attached to a support plate 30 which has the general shape of and is securely attached to the underside of the heel portion of the sole insert 12 between the sole insert 12 and the pressure switch 18. An opening or port 32 in the support plate 30 is aligned with the pressure port 20 to enable the pressure switch 18 to attach to the sole insert at the port 22 through the opening 32 in the support plate 30.

The sole insert 12 is positioned atop the sole of the shoe 40 and senses the loads placed upon the insert by a user donning the shoe 40. The sole insert 12 measures the load and communicates that measurement to the pressure switch 18. The pressure switch 18 is operatively attached to a wireless transmitter 60 that transmits the load measurements to one or more electronic receivers 62 and 64. Here, by way of example, the receiver 62 is an electronic beeper or vibrator and the receiver 64 is a cellular telephone. The receiver 62 or 64 comprises a sensory alarm (not shown) or communicates with a sensory alarm (not shown) to provide the sensory warning to the user when the load exceeds a predetermined level. Of course, either of the receivers 62 and 64 can be one of a myriad of wired or wireless reception devices capable of receiving load measurements from the pressure switch 18 or directly from the pressure sensor 12 or other analog electronic pressure transmitter (not shown), including for example, a computer, a computer tablet, a portable digital assistant, or a mechanical or electronic pressure gauge.

FIG. 8 depicts a schematic of the apparatus 10, showing the associations between each of the components. Here, sole insert 12, shown by way of example as a foot operated hydraulic pump, sends a pressure signal to the adjustable pressure switch 18. When the pressure reaches a predetermined limit set in the adjustable pressure switch 18, pressure switch 18 closes to complete the electrical circuit from the battery 28 to the sensory alarm 24. The sensory alarm 24 then activates to provide an audio, vibratory or other alert or warning to the user that the weight placed upon that lower appendage has exceeded the prescribed limit.

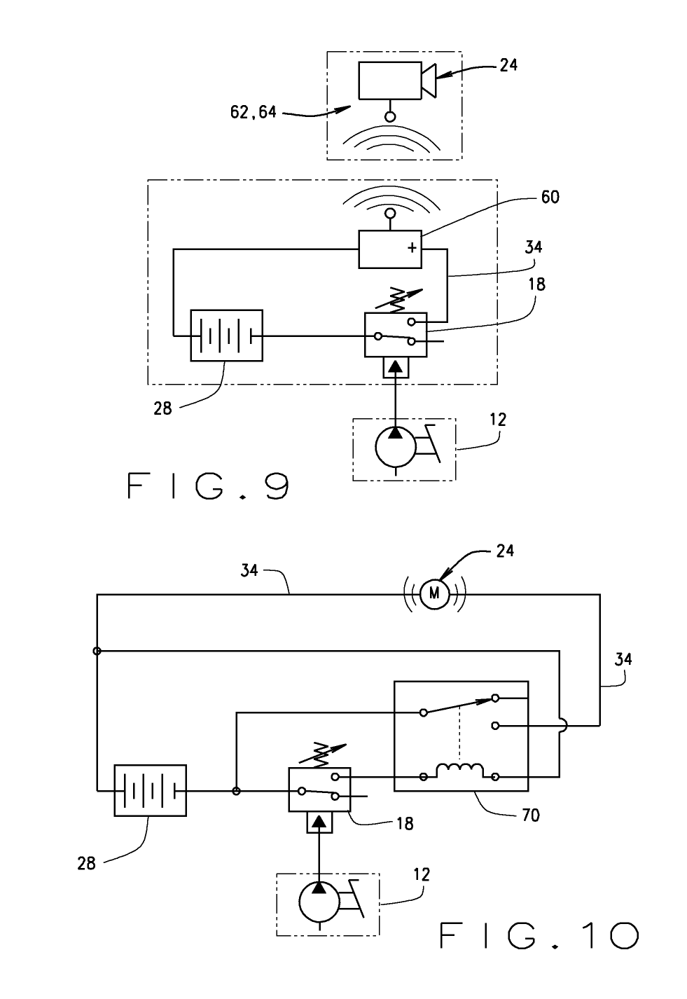

FIG. 9 depicts a schematic of a wireless electronic version of the apparatus 10, showing the electronic associations between each of the components other than the pressure sensor or sole insert 12. In this embodiment, the pressure sensor or sole insert 12 hydraulically communicates the pressure to the pressure switch 18, while the pressure switch 18 wirelessly communicates the load measurement from the pressure sensor or sole insert 12 to the sensory alarm 24 through the transmitter 60 and the receiver 62.

FIG. 10 depicts a schematic of the apparatus 10, showing the associations between each of the components. This assembly is for use when the sensory alarm requires more current than pressure switch 18 can handle. The sole insert 12 sends a pressure signal to the adjustable pressure switch 18. When this pressure reaches a predetermined limit set into the adjustable pressure switch 18, the pressure switch 18 closes to complete the electrical circuit from the battery 28 to a relay 70. This causes the relay 70 to activate, completing the circuit from the battery 28 to the sensory alarm 24. The sensory alarm 24 then activates to provide an audio, vibratory or other alert or warning to the user that the weight placed upon that lower appendage has exceeded the prescribed limit.

While I have described in the detailed description select configurations that may be encompassed within the disclosed embodiments of this invention, various other alternative configurations, that would now be apparent to one of ordinary skill in the art, may be designed and constructed within the bounds of my invention as set forth in the claims. Moreover, the above-described novel orthopedic load limit warning apparatus 10 of the present invention can be arranged in a number of other and related varieties of configurations without expanding beyond the scope of our invention as set forth in the claims.

For example, shoe insert 12 can be of varying shapes, sizes and thicknesses to fit a wide variety of footwear and to accommodate applications where the load placed upon select portions of the foot is to be measured. By way of further example, pocket 38 can have varying shapes and thicknesses, and can for example, be configured to enable the sensing of loads at specific locations on the sole insert 12.

In another example, the pressure switch 18 of the embodiment 10 can be made adjustable such that a user can set or change a desired upper load limit, and the upper load limit can be set within a range or with a margin of safety. Alternatively, the pressure switch 18 can be constructed in a variety of configurations having, for example, a single upper load limit, a specific set of predetermined upper load limits, or variable upper load limits that require a special key or code to implement. Further, the pressure switch can be electronic, digital, analog or mechanical. In addition, the entire apparatus 10 can be configured entirely, or partially, with mechanical (i.e., non-electrical or electronic) components, such as for example a complete or partial hydraulic pressure system.

Moreover, the sole insert 12 can be any of a wide variety or configuration of pressure sensing devices. Such pressure sensors can include, for example, fluid-filled chambers, peso-electronic circuitry, and pressure sensitive substrates, so long as the pressure sensor is adapted to sense a load placed upon the sensor by the lower appendage of a user and communicate that load to trigger the sensory alarm 24.

Of course, the warning apparatus 10 can be computerized. That is, the apparatus 10 can include a computer processor that controls any one or more of the warning apparatus 10 components and their interactions, including for example, the pressure sensor or sole insert 12, the pressure switch 18, the sensory alarm 24 and the battery 28.

Moreover, while I have described my invention in terms of its application to humans, I fully contemplate that the apparatus 10 can be readily adapted for use with animals. For example, the apparatus 10 can be configured with straps or other attachment devices for placement on the bottom of the leg of an animal such as a horse. In such applications, it is likely that the user will select one of the configurations of FIG. 6 or 7 with an audio sensory alarm 24 or a remote system (e.g., a receiver or cell phone 62, 64) that tracks the operation of the apparatus 10. Other similar modifications to the apparatus 10 to adapt the apparatus 10 for use with animals would be readily apparent to one of ordinary skill in the art.

When describing elements or features and/or embodiments thereof, the articles “a”, “an”, “the”, and “said” are intended to mean that there are one or more of the elements or features. The terms “comprising”, “including”, and “having” are intended to be inclusive and mean that there may be additional elements or features beyond those specifically described.

Those skilled in the art will recognize that various changes can be made to the representative embodiments and implementations described above without departing from the scope of the disclosure. Accordingly, all matter contained in the above description or shown in the accompanying drawings should be interpreted as illustrative and not in a limiting sense.

What is claimed is:

1. An orthopedic load limit warning apparatus comprising:
a. a pressure sensor shaped and sized for attachment to the underside of a user's lower appendage, the user's lower appendage placing a load upon the pressure sensor during ambulatory function by the user, the pressure sensor detecting and making a measurement of said load;
b. a pressure switch operatively associated with the pressure sensor, the pressure sensor communicating the measurement of said load to the pressure switch; and
c. a sensory alarm operatively associated with the pressure switch, the pressure switch selectively activating the sensory alarm, the sensory alarm producing a sensory warning when activated by the pressure switch;
wherein the pressure sensor activates the sensory alarm when said load measurement exceeds a predetermined level.

2. The orthopedic load limit warning apparatus of claim 1, further comprising an electric power source operatively associated with one or more of said pressure sensor, pressure switch and sensory alarm, the electric power source providing electrical current to operate said one or more of said pressure sensor, pressure switch and sensory alarm.

3. The orthopedic load limit warning apparatus of claim 2, wherein the electric power source comprises a battery.

4. The orthopedic load limit warning apparatus of claim 1, wherein the pressure sensor is shaped and sized for placement atop a sole of an article of footwear, the article of footwear being shaped and sized for placement on the base of the user's lower appendage.

5. The orthopedic load limit warning apparatus of claim 1, wherein the pressure sensor comprises a fluid-filled container.

6. The orthopedic load limit warning apparatus of claim 5, wherein the fluid-filled container comprises a sole insert shaped and sized for placement within an article of footwear.

7. The orthopedic load limit warning apparatus of claim 6, wherein the apparatus further comprises the article of footwear and the container is an integral part of the article.

8. The orthopedic load limit warning apparatus of claim 1, further comprising a computer processor operatively associated with one or more of said pressure sensor, pressure switch and said sensory alarm, the computer processor, the computer processor having computer executable instructions that control the operation of said one or more of said pressure sensor, pressure switch and said sensory alarm.

9. The orthopedic load limit warning apparatus of claim 8, wherein the computer processor generates electronic operational data from the operation of said one or more of said pressure sensor, pressure switch and said sensory alarm.

10. The orthopedic load limit warning apparatus of claim 9, further comprising a memory unit operatively associated with the computer processor, the memory unit selectively storing said operational data generated by the computer processor.

11. The orthopedic load limit warning apparatus of claim 8, wherein the computer processor comprises one of a cellular telephone, a computer, a computer tablet, and a personal organizer.

12. The orthopedic load limit warning apparatus of claim 1, wherein the pressure sensor wirelessly communicates with the pressure switch.

13. The orthopedic load limit warning apparatus of claim 1, wherein the pressure switch wirelessly activates the sensory alarm.

14. The orthopedic load limit warning apparatus of claim 1, further comprising an article of footwear shaped and sized for placement on the user's lower appendage.

15. The orthopedic load limit warning apparatus of claim 14, wherein the article of footwear comprises a heel, and one or more of said pressure sensor, pressure switch and said sensory alarm is positioned within a cavity in said heel.

16. The orthopedic load limit warning apparatus of claim 1, wherein the sensory alarm comprises one or more of an audio alarm, a tactile alarm and a visual alarm.

17. The orthopedic load limit warning apparatus of claim 1, wherein the pressure sensor electronically communicates the measurement to the pressure switch.

18. The orthopedic load limit warning apparatus of claim 1, wherein the pressure switch electronically activates the sensory alarm.

19. An orthopedic load limit warning system comprising:
a. a pressure sensor shaped and sized for attachment to the underside of a user's lower appendage, the user's lower appendage placing a load upon the pressure sensor during ambulatory function by the user, the pressure sensor detecting and making a measurement of said load, the pressure sensor generating an electronic signal indicative of the measurement;
b. an electronic data transmitter and an electronic data receiver, the transmitter and receiver operatively associated with the pressure sensor, the transmitter operatively associated with the receiver to communicate the load signal from the pressure sensor to the receiver.
c. a pressure switch operatively associated with the receiver, the receiver communicating the load signal to the pressure switch; and
d. a sensory alarm operatively associated with the pressure switch, the pressure switch selectively activating the sensory alarm when the load as indicated by said load signal exceeds a predetermined level.

20. The orthopedic load limit warning system of claim 19, further comprising a computer processor operatively associated with one or more of said transmitter and said pressure sensor, the computer processor having computer executable instructions that control the operation of said one or more of said transmitter and said pressure sensor.

21. The orthopedic load limit warning system of claim 19, further comprising a computer processor operatively associated with one or more of said receiver, pressure switch and said sensory alarm, the computer processor having computer executable instructions that control the operation of said one or more of said receiver, pressure switch and said sensory alarm.

22. The orthopedic load limit warning system of claim 19, wherein the transmitter wirelessly communicates the load signal to the receiver.

23. The orthopedic load limit warning system of claim 19, further comprising an electric power source operatively associated with one or more of said pressure sensor, transmitter, receiver, pressure switch and sensory alarm, the electric power source providing electrical current to operate said one or more of said pressure sensor, transmitter, receiver, pressure switch and sensory alarm.

24. The orthopedic load limit warning apparatus of claim 23, wherein the electric power source comprises a battery.

25. The orthopedic load limit warning apparatus of claim 19, further comprising an article of footwear shaped and sized for placement on the user's lower appendage.

26. The orthopedic load limit warning apparatus of claim 25, wherein the article of footwear comprises a heel, and one or more of said pressure sensor, pressure switch and said sensory alarm is positioned within a cavity in said heel.

27. The orthopedic load limit warning apparatus of claim 19, wherein the sensory alarm comprises one or more of an audio alarm, a tactile alarm and a visual alarm.
